(19) United States
(12) Patent Application Publication
(10) Pub. No.: US 2008/0039704 A1
(43) Pub. Date: Feb. 14, 2008

(54) Calibration of analyte measurement device

(76) Inventor: Josh N. Hogan, Los Altos, CA (US)

Correspondence Address: Josh Hogan, 620 Kingswood Way, Los Altos, CA, 94022, US

(21) Appl. No.: 11/789,277

(22) Filed: Apr. 23, 2007

Related U.S. Application Data
(60) Provisional application No. 60/798,347, filed on May 5, 2006.

(51) Int. Cl. A61B 5/00

(52) U.S. Cl. 600/365

(57) ABSTRACT

A calibration system suitable for calibrating non-invasive glucose concentration measurement systems includes one or more continuous glucose measurement devices with the ability to make continuous glucose concentration measurements at multiple regions. The system can accumulate measurement data over period of time and can process the accumulated measurement data to generate calibration related data which is used to adjust the values of one or more parameters in at least one glucose measurement device to achieve and maintain accurate performance of the continuous glucose concentration measurement device.

### CROSS REFERENCES TO RELATED APPLICATIONS

This utility patent application JH070422US claims priority from provisional application No. 60/798,347 (docket number JH060505P), filed on 5th. May 2006 the entirety of which is incorporated by reference as if fully set forth herein.

### FIELD OF THE INVENTION

The invention relates to calibration of analysis devices and in particular to calibration of continuous analysis systems that perform quantitative analysis of concentrations of specific components or analytes in targets. Such analytes include metabolites, such as glucose and such targets include tissue and blood. Such analysis devices include invasive and non-invasive devices.

### BACKGROUND OF THE INVENTION

The teaching of this application relates to subject matter in companion U.S. utility application Ser. 11/048,694 filed on Jan. 31, 2005 titled “Frequency Resolved Imaging System” which is a continuation in part of U.S. utility application Ser. No. 11/025,698 filed on Dec. 29, 2004 titled “Multiple reference non-invasive analysis system”, the contents of both of which are incorporated by reference as if fully set forth herein. This application also relates to utility patent application Ser. No. 10/870,121 filed on Jun. 17, 2004 titled “A Non-invasive Analysis System”, the contents of which are incorporated by reference as if fully set forth herein. This application also relates to U.S. utility patent Ser. No. 11/254,965 filed on 19th. Oct. 2005 titled “Correlation of concurrent non-invasively acquired signals” the contents of which are incorporated herein by reference as if fully set forth herein.

Non-invasive analysis is a valuable technique for acquiring information about systems or targets without undesirable side effects, such as damaging the target or system being analyzed. In the case of analyzing living entities, such as human tissue, undesirable side effects of invasive analysis include the risk of infection along with pain and discomfort associated with the invasive process.

In the particular case of measurement of blood glucose levels in diabetic patients, it is highly desirable to measure the blood glucose level frequently and accurately to provide appropriate treatment of the diabetic condition as absence of appropriate treatment can lead to potentially fatal health issues, including kidney failure, heart disease or stroke. A non-invasive method would avoid the pain and risk of infection and provide an opportunity for frequent or continuous measurement.

Non-invasive glucose analysis based on several techniques have been proposed. These techniques include: the technique described in “A Non-invasive Analysis System” patent application that is incorporated into this application by reference; the technique described in “Multiple reference non-invasive analysis system” patent application that is incorporated into this application by reference; optical coherence tomography (OCT), using a sequentially scanning reference mirror, as described in Proceedings of SPIE, Vol. 4263, pages 83-90 (2001).

Other techniques include various spectroscopic techniques such as: near infrared spectroscopy using both transmission and reflectance; spatially resolved diffuse reflectance; frequency domain reflectance; fluorescence spectroscopy; polarimetry and Raman spectroscopy. Other techniques, such as “photo-acoustic analysis” are described in scientific literature.

These techniques typically require a method of calibrating the measurements made by the analysis system in order to obtain an accurate glucose concentration measurement. A typical calibration approach is to compare measurements made by the non-invasive analysis system with simultaneous or concurrent measurements made by means of a conventional invasive glucose analysis system. Such conventional blood glucose analysis systems typically draw blood from the diabetic patient and directly analyze the glucose concentration of the drawn blood using conventional techniques.

Accurate invasive glucose analysis systems exist, however, they are less desirable because they are invasive and may require substantial amounts of blood, need trained personnel and are typically only available in medical institutions. These systems therefore are not suitable for frequent calibration which may be required because of slow but continuous physiological changes that can affect calibration. Some also require a costly, painful and inconvenient procedure at a medical institution or doctors office.

Readily available consumer invasive glucose analysis devices are typically based on pricking a finger to draw a small amount of blood and analyzing the drawn blood by means of a low cost glucose monitor. These consumer invasive glucose devices, however, are not very accurate and therefore if used for calibration, may require frequent comparisons to average out the random inaccuracies of the invasive glucose analysis device. Systematic inaccuracies, however, would not be removed and in any event there is the undesirable pain and inconvenience associated with frequent use of the invasive device. Indwelling and implanted consumer invasive glucose analysis devices are available, however, these devices are also typically less accurate and have their own calibration issues.

These aspects of conventional calibration approaches make them unsuitable for low cost painless, convenient and accurate calibration of continuous blood glucose concentration measurement devices. Therefore there is an unmet need for a low cost, convenient and accurate method of calibrating continuous glucose concentration measurement devices.

### SUMMARY OF THE INVENTION

The invention provides a method, apparatus and system for calibrating continuous glucose concentration measurement devices. The invention includes a continuous glucose measurement device with the ability to make continuous glucose concentration measurements at multiple regions and to process the multiple measurements to generate calibration related data which is used to achieve and maintain accurate performance of the continuous glucose concentration measurement device.

### BRIEF DESCRIPTION OF THE DRAWINGS

FIG. 1 is an illustration of the calibration system according to the invention.

FIG. 2 is an illustration of typical glucose level response profiles.

FIG. 3 is an illustration of steps in calibrating a glucose measurement device according to the method of the invention.

### DETAILED DESCRIPTION OF THE INVENTION

Some continuous glucose measurement techniques including, but not limited to, the techniques described in “A Non-invasive Analysis System” and in “Multiple reference non-invasive analysis system” patent applications that are incorporated into this application by reference, have the capability of measuring glucose concentration in various fluids including blood, intracellular fluid and interstitial fluid. For purposes of this application glucose concentration is also referred to as glucose level and intracellular fluid and interstitial fluid are referred to as tissue fluids.

To appropriately treat diabetics it is desirable to measure the glucose level of their blood, however it has been well established that the glucose level of tissue fluids are related to blood glucose level. In the steady state tissue fluid glucose levels accurately correlates with blood glucose levels. When blood glucose levels vary there is a temporal offset between blood glucose levels and tissue fluid levels. The nature of the offset depends on the nature of the blood glucose variation.

When the blood glucose level is rising the glucose level of tissue fluids typically has a temporal lag offset (or time lag) with respect to the blood glucose level. When the blood glucose level is falling the falling glucose level of tissue fluids typically has a lead offset in time with respect to the blood glucose level. Furthermore, the magnitude of the offsets typically depend on the rate of change of the blood glucose level and the magnitude of the blood glucose level. For purposes of this invention, such temporal lag or lead offsets are referred to as temporal offsets.

The present invention is a method, apparatus and system for calibrating a continuous glucose level measurement device by continuously and concurrently measuring glucose levels in different regions that can include regions of blood and regions of tissue and are herein referred to as different regions. This enables accumulating data related to: glucose levels in different regions; glucose level offsets between different regions; and the rates of change of such data. Such data contains differences in glucose levels that vary with glucose concentration. This data is accumulated over an extended period of time and is herein referred to as accumulated data. The accumulated data is correlated and processed to generate data that is used to calibrate the glucose measurement device.

The correlation and processing algorithms used to calibrate the glucose measurement device can include a model or partial model of the mechanisms causing the time offsets and correlating measured offsets with actual concurrent direct measurements of glucose levels in the various fluids in a large set of circumstances. The large set of circumstances can include multiple measurements from the same individual from multiple regions at multiple times and similar measurements from multiple individuals.

Correlation algorithms do not necessarily require an accurate understanding or description of the mechanism that causes the measured offsets. Various adaptive or learning algorithms can be used to ensure correlation and processing algorithm accurately calibrate the glucose measurement device. For purposes of this application continuously measuring means measuring at a frequency with a period that is shorter or comparable to the time period of physiological changes. Also for purposes of this application concurrently measuring includes both simultaneously measuring and measuring within a time period that is short in relation to the time period of physiological changes.

The frequency with which actual direct measurements are made concurrently to provide correlation data can be reduced or increased depending on the accuracy with which the measured data, including the temporal offset data, correlates with the accumulated data and with actual direct measurement data. The accuracy of correlation is used to generate a figure of merit which can be used to indicate the frequency with which direct glucose measurements should be made.

For example, a diabetic patient using a conventional (blood drawing and test strip) direct glucose measurement system could transition to a continuous non-invasive glucose measurement system by gradually reducing the frequency of conventional direct glucose measurements as the continuous device achieves good calibration. During initial use of the continuous glucose level measurement system the diabetic could continue to make direct glucose measurements with their customary frequency.

During this initial period the continuous glucose measurement device will accumulate measurement data and receive (as input data from the diabetic or from his monitor) the results of the direct measurements. The accumulated data and direct measurement data is processed and correlated. Processing and correlation can include matching temporal offset data and trend data with the direct measurement data and refining a specific calibration model or algorithm.

As good correlation is achieved, indicated by the correlation figure of merit, the frequency with which direct measurements are taken can be reduced. With a continued good correlation figure of merit, direct measurements can be made very infrequently. Very infrequent direct measurements allow the infrequent direct measurements to be made under more controlled conditions, such as at a very specific time with respect to meals and exercise and selected by the calibration process.

Very infrequent direct measurements also make it realistic to make more accurate direct measurements by drawing a larger blood sample and using higher quality and more complex test strips. Very infrequent direct measurements can also enables using more complex and more accurate test strips and monitors (e.g. at a pharmacy) and having test strips processed remotely.

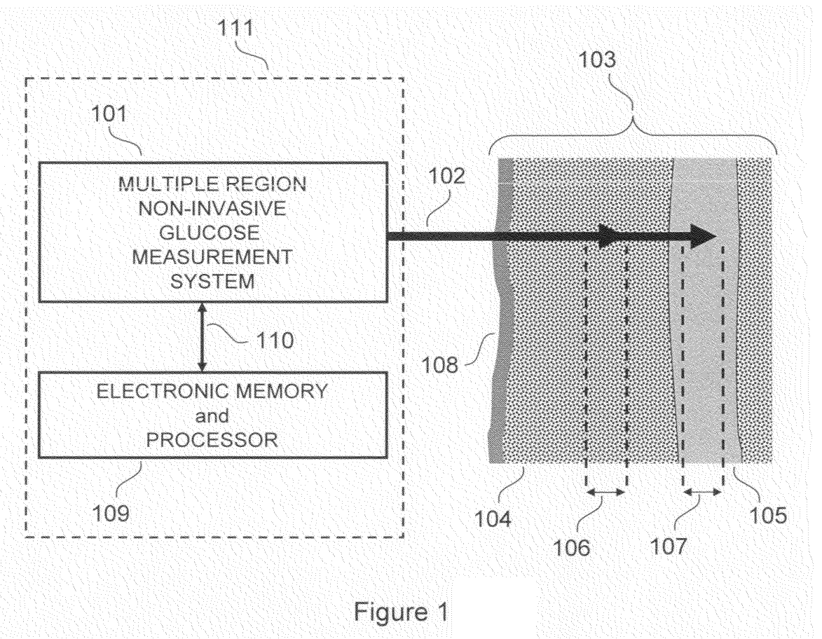

A preferred embodiment of this invention is illustrated in and described with reference to FIG. 1 where a multiple region non-invasive optical analysis device 101 applies an optical probe beam 102 to a target 103 consisting of tissue 104 and blood 105. The optical analysis device 101 is operated to analyze two regions along the path of the probe beam 102 of a target concurrently. One region, located in tissue 104 is indicated by the double arrow 106 and its associated dashed lines. The other region, located in blood 105 is indicated by the double arrow 107 and its associated dashed lines.

An advantage of using a non-invasive glucose measurement systems based on the techniques described in “A Non-invasive Analysis System” and in “Multiple reference non-invasive analysis system” patent applications that are incorporated into this application by reference is that these techniques provide a high degree of insensitivity to environmental and physiological changes at the outer region 108 or skin of the tissue.

The optical analysis device 101 analyses the backscattered radiation from the two regions (indicated by 106 and 107) to determine the glucose levels in these regions concurrently and continuously. The optical analysis device 101 makes the glucose level information available to an electronic memory and processor 109 which may be either a local or remote electronic memory and processor. In the case of a local electronic memory and processor the units enclosed in the dashed box 111 of FIG. 1 would constitute an integrated self-calibrating monitor.

The electronic memory and processor 109 is connected to the optical analysis device 101 by means of an electronic interface 110 which, in the case of a remote system, may be a wireless interface to the Internet or a wired interface, such as USB (Universal Serial Bus) to an Internet connected PC (Personal Computer).

An advantage of a remote electronic memory and processor is that calibration processing can avail of correlating a single individual's data with a large accumulated set of calibration data and extensive processing using complex algorithms, including adaptive or learning algorithms, to generate calibration information. The large accumulated set of calibration data can include data from similar individuals. Similar individuals include individuals at a similar stage of diabetes, with similar lifestyle and have other similar characteristics that are relevant to the processing of glucose or insulin by the individuals.

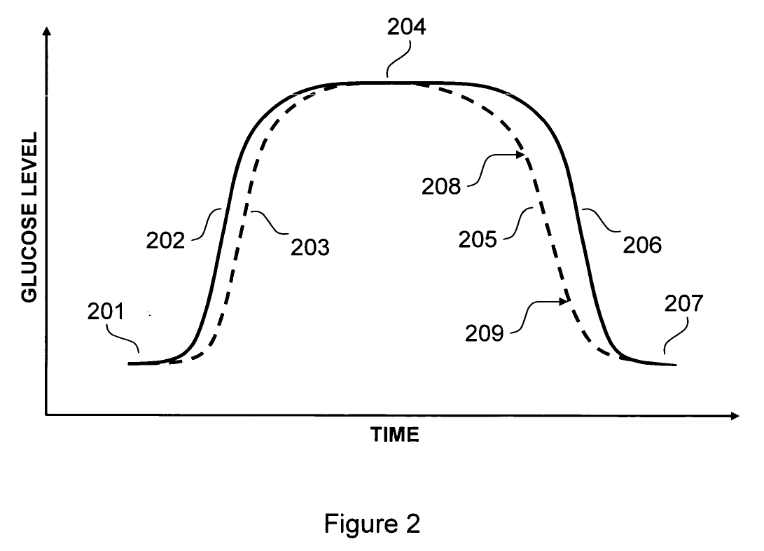

Measuring glucose levels at two regions concurrently enables measuring temporal offset data. When glucose levels are changing there can be a temporal offset for different regions to achieve the same glucose level. This is illustrated in FIG. 2 where a typical variation with time of blood glucose level and tissue fluid glucose level are shown.

Both the blood glucose level and tissue fluid glucose level have an initial low level 201. In a typical response to ingesting food glucose levels rise, but with different response times. The blood glucose level 202 rises faster than the tissue fluid glucose level 203. As glucose levels stabilize the blood glucose level and tissue fluid glucose level achieve the same level 204.

As insulin is absorbed, glucose levels fall. The tissue fluid glucose level 205 can fall at a different rate than the blood glucose level 206 and again achieve the same level 207 as they stabilize. The magnitude of the temporal offset between the blood glucose level and tissue fluid glucose level can vary with the actual glucose level. This is illustrated by the large temporal offset 208 at a high glucose level and the smaller temporal offset 209 at lower glucose levels.

The temporal offset information acquired by concurrently measuring glucose levels in two different regions can thus be used to gain information about the actual glucose concentration level. This information is accumulated over a long period of time and correlated with previously generated and accumulated data and direct measurement data. Processing of such data can include refining a model or algorithm that correlates well with the glucose level response in different regions. Previously generated data can include data generated by other monitors and related to other individuals.

Different regions include at least two different regions. Different regions include; one containing tissue fluid and one containing blood; one containing tissue fluid and another also containing tissue fluid and substantially at the same location on the body but at a different depth with respect to the skin surface; one containing tissue fluid and another also containing tissue fluid and at a substantially different location on the body; and one containing blood and another also containing blood and at a substantially different location on the body.

Processing and correlating accumulated data, direct measurement data, and data from a refined model or algorithm provides correlation data that is used to adjust parameters such as the gain and offset of the continuous non-invasive glucose measurement system and thereby calibrate the system. For purposes of this invention, “parameters” includes but is not limited to conventional electronic gain and offset values; tables of values, such as PROM tables; and coefficients of algorithms. Calibration data will be used to adjust such parameters by electronic circuitry in the monitor that has access to the calibration data and modifies the monitor in its measurement of glucose levels. Calibration will typically be implemented using data accumulated over significant periods of time and changes will be applied slowly to reduce sensitivity to random or unusual effects and provide smooth calibration corrections.

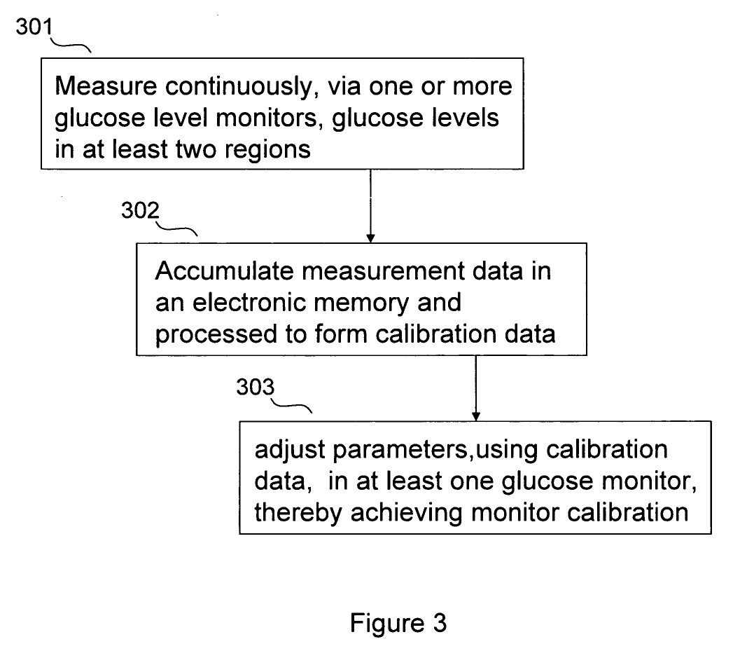

FIG. 3 illustrates steps of 301 continuously measuring glucose levels using one or more glucose level monitors in at least two regions, then 302 accumulating measurement data is in an electronic memory and processing the measurement data to form calibration data, and then 303 using the calibration data is to adjust parameters in at least one glucose monitor to achieve monitor calibration.

The figure of merit (indicating the accuracy of calibration) can be derived from the magnitude and degree of variation in parameter adjustments required to achieve calibration and will be monitored to provide guidance on the frequency with which (if ever) conventional direct blood glucose measurements should be made to ensure the continuous non-invasive glucose measurement system remains appropriately calibrated.

Calibration can be facilitated by ingesting specific nutrients (foods, drinks, etc) under defined conditions of environment, for example a resting period for a specified time period. Specific nutrients include, but are not limited to: nutrients with a known glucose content; nutrients that contain known quantities of embedded insulin in one form or another; nutrients that contain known quantities of glucose and known quantities of embedded insulin in one form or another; and nutrients that contain known quantities of embedded insulin in one form or another with known timed release characteristics.

It is understood that the above description is intended to be illustrative and not restrictive. Many of the features have functional equivalents that are intended to be included in the invention as being taught.

Many variations and combinations of the above embodiments are possible, for example, multiple continuous non-invasive glucose measurement systems could be used to measure glucose levels at different locations on the body. Individual continuous non-invasive glucose measurement systems could measure at adjacent locations, but be optimized for different depths or different environments, such as one optimized for interstitial fluid glucose level measurement and one optimized for blood glucose level measurement.

Continuous indwelling (sub-cutaneous) or implanted glucose measurement devices could be used. Glucose monitors based on various technologies could be used. Such technologies include, but are not limited to: other forms of OCT, such as Fourier or spectral domain OCT; photo-acoustic effect; spectroscopic technologies; and electro-chemical technologies.

Rates of change of glucose levels and rates of change of glucose level offsets can be included in processing and correlation. Parameters relating to environment, such as time of day, season time, temperature, humidity, can be included in processing and correlation. Parameters relating to behavior, such as relaxation, exercise, travel, can be included in processing and correlation.

Many of the features have functional equivalents that are intended to be included in the invention as taught. For example, measuring devices could be wirelessly connected to a separate processing unit. Other examples will be apparent to persons skilled in the art. The scope of this invention should be determined with reference to the specification, the drawings, the appended claims, along with the full scope of equivalents as applied thereto.

What is claimed is:

1. A method for calibrating a glucose level measurement device comprising:
measuring glucose levels in at least two different regions to form measurement data;
accumulating said measurement data over a period of time to form accumulated data;
processing said accumulated data to generate calibration data; and
using said calibration data to calibrate said glucose level measurement device.

2. The method of claim 1, wherein the glucose measurements at different regions are made concurrently.

3. The method of claim 1, wherein the glucose measurements at different regions are made continuously.

4. The method of claim 1, wherein the different regions are different depths within tissue.

5. The method of claim 1, wherein the different regions are tissue at different locations of the body.

6. The method of claim 1, wherein the different regions differ by one region being tissue and another region being blood.

7. The method of claim 1, wherein the accumulated measurement data is processed by correlating it with previously acquired measurement data.

8. The method of claim 1, wherein the accumulated measurement data is processed to determine temporal offset data.

9. The method of claim 8, wherein the temporal offset data is correlated with previously generated measurement data to generate calibration data.

10. The method of claim 1, wherein the calibration data is used to adjust parameters of a glucose level measurement device.

11. The method of claim 1, wherein the glucose level measurement device is a continuous glucose level measurement device.

12. The method of claim 1, wherein the glucose level measurement device is a continuous non-invasive glucose level measurement device.

13. A system for calibrating a glucose level measurement device, said system comprising:
at least one monitor, said monitor containing electronic circuitry, operable to measure glucose levels in at least two different regions to form measurement data;
an electronic memory operable to accumulate said measurement data over a period of time to form accumulated data;
a processor operable to process said accumulated data to generate calibration data; and
said electronic circuitry operable to access said calibration data and operable to adjust parameters depending on said calibration data to modify said monitor in its measurement of glucose levels whereby calibration of said glucose level measurement device is achieved.

14. An apparatus for calibrating a glucose level measurement device comprising:
means for measuring glucose levels in at least two different regions to form measurement data;
means for accumulating said measurement data over a period of time to form accumulated data;
means for processing said accumulated data to generate calibration data;
means for using said calibration data, wherein said use enables calibration of said glucose level measurement device.

15. The apparatus of claim 14, wherein the glucose measurements at different regions are made concurrently.

16. The apparatus of claim 14, wherein the glucose measurements at different regions are made continuously.

17. The apparatus of claim 14, wherein the different regions are different depths within tissue.

18. The apparatus of claim 14, wherein the different regions are tissue at different locations of the body.

19. The apparatus of claim 14, wherein the different regions differ by one region being tissue and another region being blood.

20. The apparatus of claim 14, wherein the accumulated measurement data is processed by correlating it with previously acquired measurement data.

21. The apparatus of claim 14, wherein the accumulated measurement data is processed to determine temporal offset data.

22. The apparatus of claim 21, wherein the temporal offset data is correlated with previously generated measurement data to generate calibration data.

23. The apparatus of claim 14, wherein the calibration data is used to adjust parameters of the glucose level measurement device.

24. The apparatus of claim 14, wherein the glucose level measurement device is a continuous glucose level measurement device.

25. The apparatus of claim 14, wherein the glucose level measurement device is a continuous non-invasive glucose level measurement device.
